(19) United States
(12) Patent Application Publication
(10) Pub. No.: US 2013/0165877 A1
(43) Pub. Date: Jun. 27, 2013

(54) AUGMENTED CHEST TUBE DRAINAGE SYSTEM AND METHOD WITH VOLUME SENSING, AUTOMATED ALERTS AND MESSAGING CAPABILITY

(71) Applicants: Cory E. Leeson, Pittsburgh, PA (US); Joseph Thomas Samosky, Pittsburgh, PA (US)

(72) Inventors: Cory E. Leeson, Pittsburgh, PA (US); Joseph Thomas Samosky, Pittsburgh, PA (US)

(73) Assignee: University of Pittsburgh-of the Commonwealth System of Higher Education, Pittsburgh, PA (US)

(21) Appl. No.: 13/724,904

(22) Filed: Dec. 21, 2012

Related U.S. Application Data
(60) Provisional application No. 61/578,514, filed on Dec. 21, 2011.

(51) Int. Cl. A61M 27/00

(52) U.S. Cl. CPC A61M 27/00
USPC 604/318

(57) ABSTRACT

Systems and methods that can augment conventional chest tube drainage canisters are discussed. One such system can monitor a chest tube drainage canister that collects fluid and includes a volume sensor that can determine a volume of the fluid. Such a system can also include a control component that calculates a flow rate of the fluid. The control component can compares the volume and the flow rate with one or more predetermined conditions, and the control component provides at least one notification to a healthcare professional when at least one of the predetermined conditions are met. Additionally, a user interface can be provided for monitoring fluid volume and flow rate, entering alerts capable of defining conditions for notification of healthcare professionals, and entering contact information for automatic notification of healthcare professionals via email or text message in connection with the alerts.

### CROSS-REFERENCE TO RELATED APPLICATIONS

This application claims the benefit of pending U.S. Provisional Patent application Ser. No. 61/578,514 (Atty. Dkt. No. 02643/106852.49PRO) entitled ‘PLEURALERT: AN AUGMENTED CHEST TUBE DRAINAGE SYSTEM WITH ELECTRONIC VOLUME SENSING, AUTOMATED ALERTS AND TEXT MESSAGING CAPABILITY’ and filed Dec. 21, 2011. The entirety of the above-noted application is incorporated by reference herein.

### BACKGROUND

Over one million chest tubes are deployed annually for patients in the U.S. alone. Commercially available chest tube drainage canisters require nurses to read volume via a graduated scale: nurses may need to kneel down to floor level to observe the fluid and manually mark the level and observation time on the canister with a pen. This can be time consuming, inconvenient, and subject to error. Different nurses may mark levels and times in different ways, leading to difficulties in interpreting marks, discerning trends over time or communication between shifts. Furthermore, the monitoring process is manual and only done at periodic intervals so sudden changes in chest tube output, such as may result from a sudden postoperative hemorrhage into the pleural space, may go undetected until the nurse's next check of the container. This can entail risk for the patient's safety.

### SUMMARY

The following presents a simplified summary of the innovation in order to provide a basic understanding of some aspects of the innovation. This summary is not an extensive overview of the innovation. It is not intended to identify key/critical elements of the innovation or to delineate the scope of the innovation. Its sole purpose is to present some concepts of the innovation in a simplified form as a prelude to the more detailed description that is presented later.

The innovation disclosed and claimed herein, in one aspect thereof, comprises a system that facilitates monitoring of chest tube drainage. Such a system can include a chest tube drainage canister that collects fluid and a volume sensor that can determine a volume of the fluid. The system can also include a control component that calculates a flow rate of the fluid. The control component can compare the volume and the flow rate with one or more predetermined conditions, and the control component provides at least one notification to a healthcare professional when at least one of the predetermined conditions are met. Additionally, a user interface can be provided for monitoring fluid volume and flow rate, entering alerts capable of defining conditions for notification of healthcare professionals, and entering contact information for automatic notification of healthcare professionals via email or text message in connection with the alerts.

In another aspect, the subject innovation can include a method of monitoring a chest tube drainage canister. Such a method can include the acts of measuring a volume of a fluid in the chest tube drainage canister, determining a flow rate of the fluid in the chest tube drainage canister, and comparing the volume and the flow rate to one or more predetermined conditions. Additionally, the method can include the acts of determining whether at least one of the one or more predetermined conditions has been met, receiving contact information associated with a healthcare professional, and providing at least one notification to the healthcare professional when the at least one of the one or more predetermined conditions has been met. The notification can include information indicating which predetermined condition has been met.

To the accomplishment of the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

FIG. 1 illustrates a chest tube drainage system in accordance with aspects of the subject innovation.

FIG. 2 illustrates an example sequence of events from a screenshot of a prototype system of the subject innovation.

FIG. 3 illustrates a method of monitoring a chest tube drainage canister in accordance with aspects of the innovation.

FIG. 4 illustrates hardware components associated with an embodiment of the subject innovation.

FIG. 5 illustrates actions of software components associated with an embodiment of the subject innovation.

FIG. 6 illustrates an embodiment of a system of the subject innovation attached to a standard IV pole.

FIG. 7A illustrates an example user interface of a system of the subject innovation.

FIG. 7B illustrates a graph of fluid volume from an example user interface of a system of the subject innovation.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

The subject innovation relates to improvements to a conventional medical device, the chest tube drainage canister. These improvements can include, for example, the addition of electronic sensing of fluid volume, automated detection of critical alarm conditions, and the ability to automatically send alert text messages to a nurse's cell phone. Systems of the subject innovation can provide a user interface with multiple features not available in conventional systems, for example, a touch-screen interface and the ability to graphically display chest tube output over time. The subject innovation augments a device whose basic function dates back 50 years by adding features that can automate and optimize a monitoring process that can be time consuming and inconvenient for nurses. The system may also enhance detection of emergency conditions and speed response time.

Many medical devices and systems designed in recent years are smart, interconnected medical devices with sensing, automated decision support and networked communication capabilities. Unfortunately, many commonly used medical devices, such as chest tube drainage canisters, do not yet have such capabilities. The subject innovation improves on conventional chest tube drainage containers by providing a system with additional features described herein, for example, electronic sensing, automated alerts and text messaging capabilities.

Over one million chest tubes are deployed annually for patients in the U.S. alone. Commercially available drainage canisters require nurses to read volume via a graduated scale: nurses may need to kneel down to floor level to observe the fluid and manually mark the level and observation time on the canister with a pen. Furthermore, emergency conditions such as a sudden postoperative hemorrhage into the pleural space may go undetected until the nurse's next check of the container.

As described supra, conventional chest drainage canisters are purely mechanical devices. The subject innovation, however, includes systems and methods that can work in conjunction with many currently available chest drainage containers to automatically monitor and display the volume of fluid effluent, detect alarm conditions, and automatically generate alerts.

Traditional chest drainage systems have no method of alerting a nurse to a critical alarm condition. In aspects, the subject innovation can have a plurality of alerts (e.g., high volume, high flow rate, low flow rate, etc.) wherein each alarm state can trigger alerts, e.g., text messaging alerts or emails sent to a nurse (e.g., via a cell phone or other email-enabled or SMS-capable devices).

In aspects, the subject innovation can continually, repeatably and objectively measure chest drainage and present this data in quickly and easily comprehendible graphical displays. As will be appreciated, aspects of the subject innovation can provide for multiple advantages over the current system of manual checking, marking levels with pens and tape, and seeing only hand-written numeric data to evaluate chest fluid output over time.

The subject innovation can function as a weight-based system, using an estimate of the density of physiological fluids drained from the pleural space to calculate the volume of fluid in the chest drainage canister. Because the weight sensing unit is completely isolated from body fluids (which are wholly contained in the canister), the system can be reusable, reducing costs for expendables.

Additionally, the entire system can adjustably mount on an IV pole to ensure the drainage canister is kept below chest level for gravity drainage of fluid. Users can interact with the system via a user interface that can include a compact, touch-screen interface. Users can have the option to view a chart display screen that allows a physician or nurse to see a real-time trend of fluid volume over time and allows the user to select various time intervals to determine how the patient's fluid volume is trending.

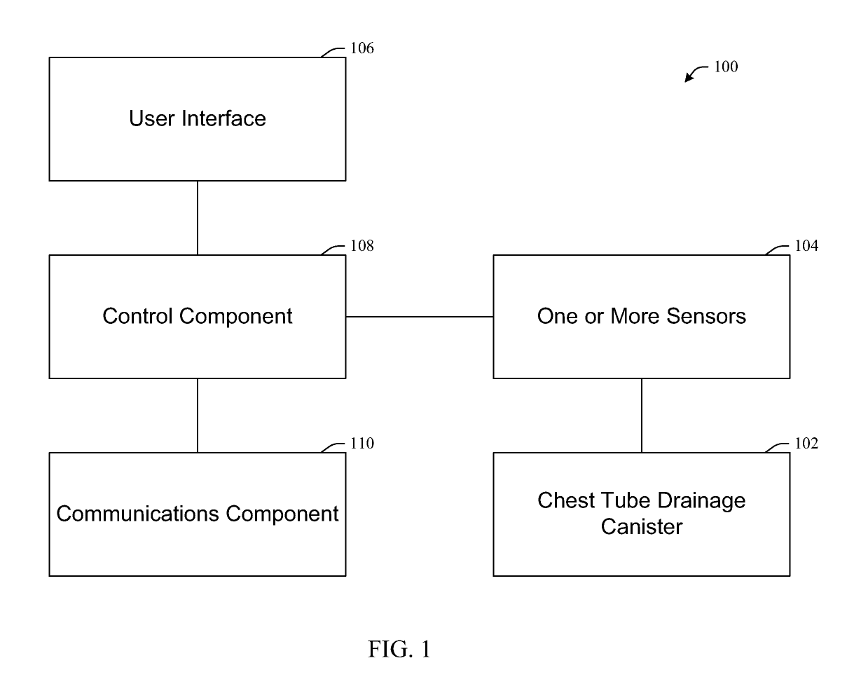

Referring initially to the drawings, FIG. 1 illustrates a chest tube drainage system 100 in accordance with aspects of the subject innovation. System 100 can include a chest tube drainage canister 102 to collect fluid drained from a patient via a chest tube. The canister can be, for example, of any of a variety of conventional designs.

One or more sensors 104 can monitor properties of chest tube drainage canister 102 or other components of system 100. These one or more sensors 104 can include a force sensor that can measure the weight of fluid in drainage canister 102, from which an estimate of the volume of fluid in drainage canister 102 can be obtained as described herein. Additional sensors can also be included. For example, as described herein, the subject innovation can be attached to a pole used for intravenous therapy (IV pole) and can be operated on battery power to allow certain patients to walk around as appropriate. The one or more sensors 104 can include a sensor to monitor the current level of battery power remaining, such that an alert can be provided before loss of power. Additionally, a sensor for determining the location of the system 100 (e.g., via global positioning system (GPS), radio frequency identification (RFID) via proximity to one or more RFID readers at known locations, etc.) can be included such that if a patient needs to be found (e.g., due to an emergency situation, etc.), such information can be included in an alert.

System 100 can further include a user interface 106 through which a user can interact with system 100. User interface 106 can provide for the ability to view data collected by the one or more sensors 104 in a variety of ways, such as current values, historical trends, graphical displays, etc. Additionally, user interface 106 can display alert information, such as showing one or more active alerts, a log of prior alerts, etc. Additionally, the user interface 106 can provide for user input (e.g., via a touch screen, etc.), allowing a user to select data to view and a manner of viewing the data (e.g., numerically or graphically (or both), or by adjusting a time interval over which data is presented to determine trends, etc.). Furthermore, the user interface 106 can provide for the ability to select premade alerts or customize alerts, which can be designed specifically to the patient, and can designate one or more notifications associated with the alert (e.g., text, email, audio, visual, etc.) and designate one or more recipients (e.g., email addresses, cell phones or other short message service (SMS)-enabled devices). These alerts can include threshold values for volume and flow rate, such as a threshold value for the volume that triggers a notification when exceeded, an upper threshold value for the flow rate that triggers a notification when exceeded, and a lower threshold value for the flow rate that triggers a notification when the flow rate falls below the lower threshold value for the flow rate.

Control component 108 can monitor the data collected by the one or more sensors 104 to generate the information presented via user interface 106. Control component 108 can include any of a variety of hardware for implementing the functions described herein, and can be, for example, a compact single-board computer with battery backup for mobile operation. Additionally, alerts or other information entered by a user via user interface 106 can be monitored in connection with the data from the one or more sensors 104, and when the condition or conditions of an alert are met, one or more notifications can be provided in the one or more pre-selected ways to a healthcare professional (e.g., nurse, physician, etc.) responsible for a patient associated with system 100, such as by sending an email, text message, etc. via communications component 110. For example, if an alert is based on a high volume, control component 108 can monitor the output from a weight sensor of the one or more sensors 104 such that a volume can be estimated. When the estimated volume reaches the level specified in the alert, one or more notifications associated with the alert can be presented (e.g., a text message to a nurse responsible for the patient can be sent via communications component 110, etc.). In aspects, if it is determined that system 100 is operating on battery power and thus might not be located in the patient's room, the notification can include location information associated with system 100.

In some aspects of the embodiment, control component 108 can wirelessly stream sensor and other data (e.g., fluid volume and alert data) directly to a patient's electronic medical record via communications component 110, which can enhance individual patient care and support research in postoperative best care practices.

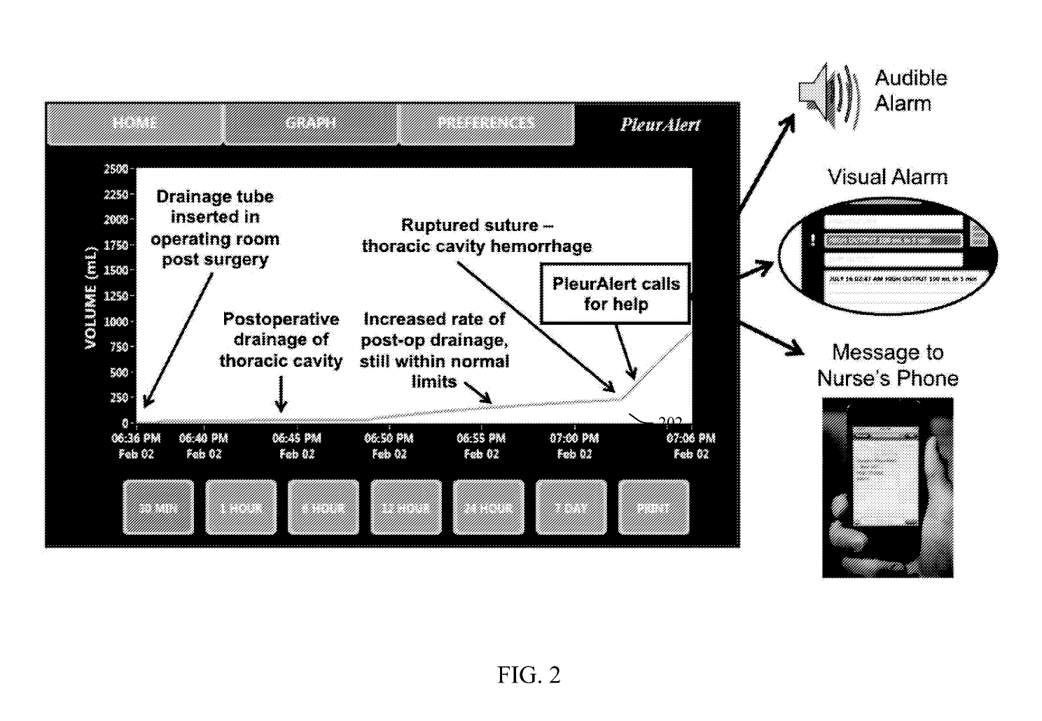

Systems and methods of the subject innovation can work in conjunction with any of a variety current chest drainage canisters to provide features described herein, such as the ability to automatically monitor and display the volume of fluid effluent. The system can have a plurality of alerts, such as alerts for high volume, high flow rate, and low flow rate. Each of these alerts can trigger text messaging alerts sent via email to a cell phone or other email-enabled or SMS-capable device if the associated condition occurs (e.g., high volume, etc.). FIG. 2 shows a screenshot of a prototype system of the subject innovation and illustrates an example sequence of events during a simulation-based test of the system (controlled fluid input, non-clinical data). At 202, when the flow rate exceeds a threshold value condition associated with an alert, a notification can be provided to one or more healthcare professionals.

The one or more sensors 104 can sense volume via a high-accuracy, low-creep strain gauge load sensor to measure the weight of the chest tube container. Weight can then be used to estimate fluid volume in the chamber. The density of physiological liquids that may be present in the pleural space range from approximately 1.010 g/mL to 1.0549 g/mL (the upper range for whole blood). In various aspects, the weight-to-volume conversion employed by systems and methods of the subject innovation can be calibrated based on a value at or near the lower bound of the density range, for example, by using the density of water, 1.0 g/mL. Because of the variation in density in physiological fluids, selecting a value at or near the lower bound can ensure that the system will not underestimate the true volume in the chamber for any potential combination of body fluids, with a worst case error for the example given (assuming whole blood) of +5.49%/−0%.

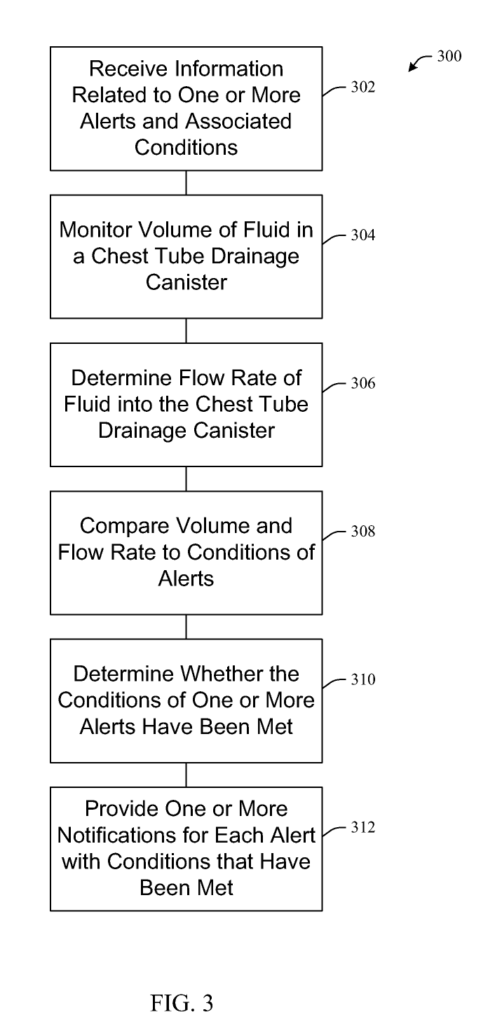

Referring now to FIG. 3, illustrated is a method 300 of monitoring a chest tube drainage canister in accordance with aspects of the innovation. While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

Method 300 can begin at 302 by receiving information related to one or more alerts. These alerts can designate one or more conditions that must be met for the alert to be triggered and a notification to be provided to a healthcare professional responsible for a patient associated with the chest tube drainage canister. Examples of such conditions are high fluid volume, high fluid flow rate, low fluid flow rate, etc. The information received can include selections of alerts to be active, values for conditions associated with the alerts (e.g., specific volume levels, flow rates, etc.), or contact information (e.g., phone number, email address, etc.) of one or more designated healthcare professionals who will receive a notification if the condition of the alert is met. Next, at 304, the volume of fluid in the chest tube drainage canister can be monitored, for example, via determining the weight of fluid in the canister and estimating the volume as described herein. At 306, a flow rate of fluid into the canister can be determined based on volume measurements and associated time data. Then, at 308, the volume and flow rate can be compared to the conditions associated with the one or more alerts, and a determination can be made at 310 whether any of those conditions have been met (e.g., volume exceeds a first value, flow rate exceeds a second value or falls below a third value, etc.). If one or more of the conditions have been met, then at 312, the one or more healthcare professionals can be provided notifications based on the information received at 302.

What follows is a more detailed discussion of certain systems, methods, and apparatuses associated with aspects of the subject innovation. To aid in the understanding of aspects of the subject innovation, theoretical analysis and experimental results associated with specific experiments that were conducted are discussed herein. However, although for the purposes of obtaining the results discussed herein, specific choices were made as to the selection of various aspects of the experiments and associated setups—such as specific choices of sensors and layouts of interfaces—the systems and methods described herein need not conform to the specific embodiments discussed herein. For example, various aspects of the subject innovation can be utilized to monitor volume or flow rate of fluid based on weight determinations. In various embodiments, however, different components or sensors can be selected than those used in the experiments discussed herein (e.g., a fluid level sensor or other sensor could be employed to determine volume, etc.), as explained in greater detail below.

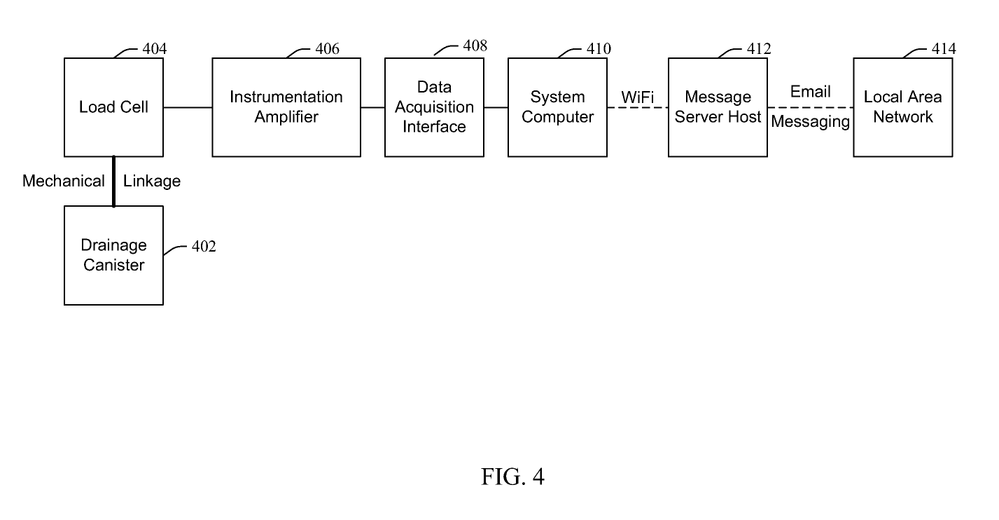

FIG. 4 illustrates hardware components associated with an embodiment of the subject innovation capable of use in connection with a chest tube drainage canister 402. Such a system can include a weight or volume sensor 404 such as a high-accuracy, low-creep strain gauge load sensor that can be used to measure the weight of the chest tube canister 402. The analog output of the load cell 404 can be amplified and instrumentation amplifier 406 and digitized by a data acquisition interface 408. System computer 410 can filter the load signal, tare the initial weight of the empty chamber, monitor increases in weight, convert weight increments to fluid volume, automatically detect alarm conditions, triggered alerts, generate the user interface and connect wirelessly to the local network (e.g., using Microsoft's .NET framework). In the event of an alarm condition or triggered alert, system computer 410 can provide a notification via WiFi to a message server host 412 that can then send an email to the local area network 414 for notification of a healthcare provider responsible for the patient associated with the system. Other modes of notification can be used additionally or alternatively, such as text messaging, visual alarms, audio alarms, etc.

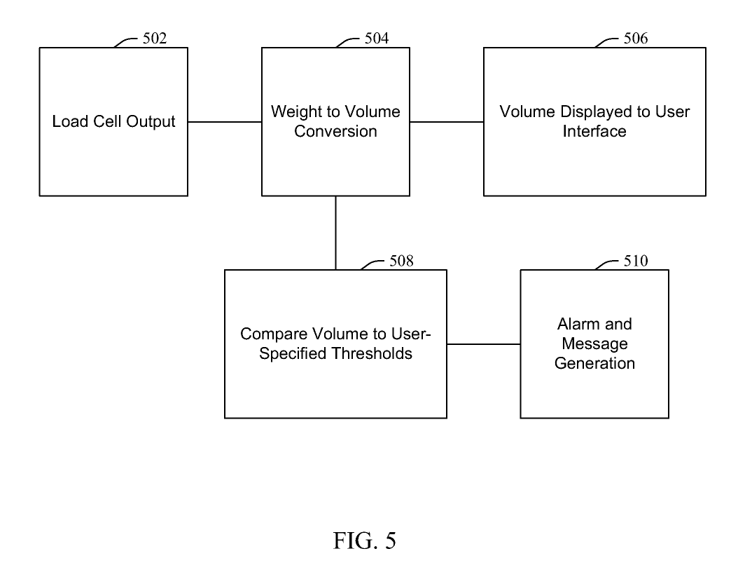

FIG. 5 illustrates actions of software components associated with an embodiment of the subject innovation. The load cell output can be received as shown at 502, indicating the weight of the chest tube drainage canister. As seen at 504, weight to volume conversion calculations can be performed to estimate the volume of fluid in the chest tube drainage canister. This volume information can be displayed via a user interface as in 506. The display can be one or more of numerical, a virtual tank display, a chart, etc. The user interface can also display alarm information, such as displaying active alarms, an alarm history log, etc. Additionally, as seen at 508, the volume from 506 can be compared to one or more user-specified thresholds associated with alerts. If any of the thresholds for the alerts have been met, then corresponding alarms and notification messages can be generated at 510. These alarms and or notifications can include visual alarms, auditory alarms, text messages, email messages, etc.

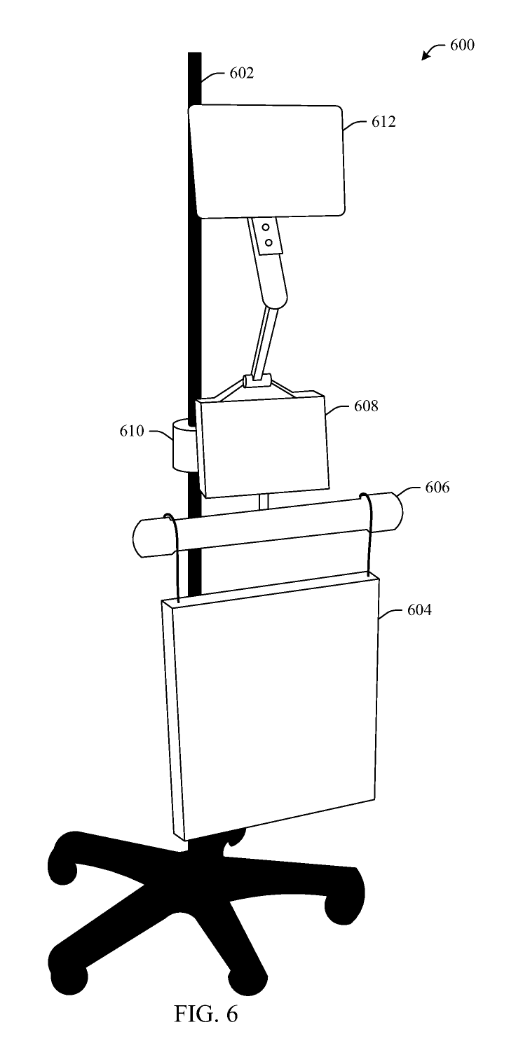

FIG. 6 illustrates an embodiment of a system 600 of the subject innovation attached to a standard IV pole 602. The conventional drainage canister 604 can be hung on a horizontal post 606 that is connected via a mechanical linkage to a load cell (or other weight sensor) in the main system control and electronics module 608. The analog output of the load cell can be amplified, digitized and input into a program that can tare the initial weight of the empty chamber, measure increases in weight, convert weight increments to fluid volume, automatically detect alarm conditions, generate the user interface and connect wirelessly to the local network. The main system module 608 can be secured to IV pole 602 via an adjustable pole mount 610.

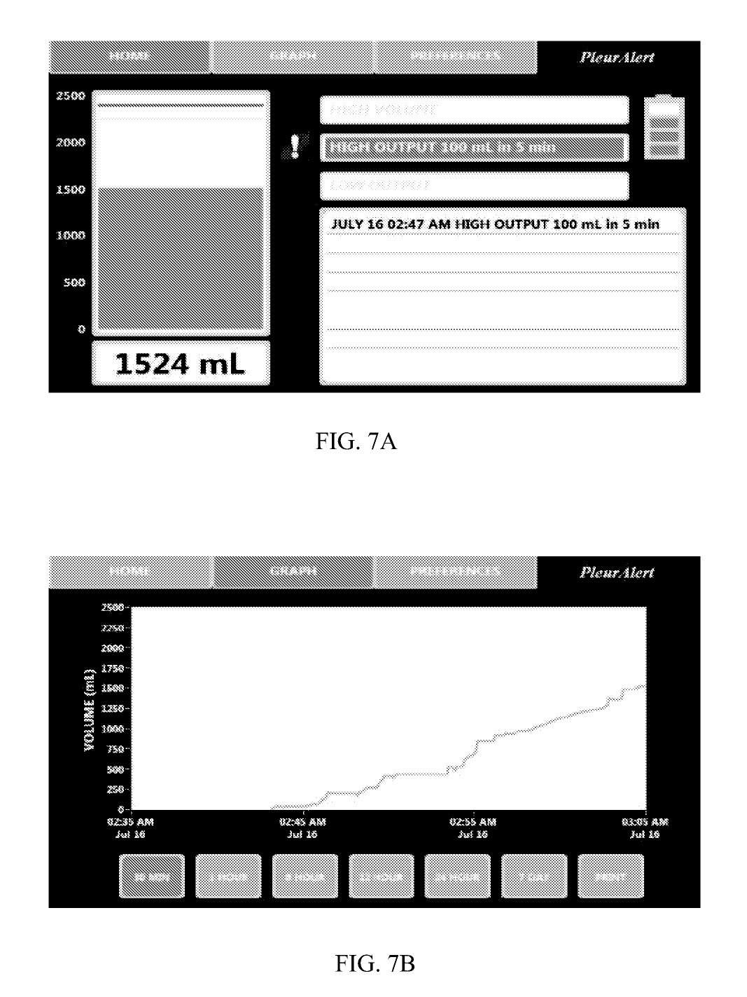

Users can interact with system 600 via a touch-screen interface 612. The screen can have a default window which can be a main display presenting one or more items of useful information. FIG. 7A illustrates an example of such a default window, where current fluid volume, battery life, alarm history and visual alarms are shown. FIG. 7B illustrates an example of a graphical or chart display of volume data, allowing a healthcare professional (e.g., physician or nurse) to see a real-time trend of fluid volume over time and enabling the user to select various time intervals (in the chart, 30 minutes has been selected) to understand how the patient's fluid volume is trending. A configuration screen can be included to allow the user to input patient-specific alarm thresholds and designate who the recipients of the text messages, emails, etc. will be.

An initial prototype system (PleurAlert) was built and successfully measured water volume added to the drainage chamber. Alerts were generated appropriately, as shown in FIG. 7A, according to preset threshold conditions and the system sensed, recorded and displayed the volume in both numerical and graphical formats, such as the graph illustrated in FIG. 7B. Additionally, the prototype was capable of automated generation and wireless transmission of alert text messages to a cell phone.

Additionally, experiments were performed to determine sensitivity of volume to motion of the system when mounted on a wheeled IV pole. Initial tests on an early prototype showed that the system had a maximum percent change in sensed volume of less than 1% when the system was in motion across a flat surface at standard walking speed. To prevent triggering of possible false alarms when the system is subjected to larger accelerations which might cause transient spurious peaks in the volume measurement (such as might be encountered if the IV pole is rolled across a bump or step) an accelerometer can be incorporated as an additional sensor into the system. Triggering of alerts can then be transiently suppressed when high system accelerations are detected.

Systems and methods of the subject innovation are designed to enhance, rather than replace, a familiar medical device that is universally deployed in hospitals. In cases of a ruptured suture or blown graft, hemorrhaging patients can lose a liter or more of blood within minutes. Sudden stoppage of drainage is indicative of a clotted line—unless dealt with immediately, the chest cavity may quickly fill with fluid and cause life-threatening cardiac tamponade. Embodiments of the subject innovation offer the potential to automatically detect such conditions and call for help. The subject innovation also offers the capability for wireless streaming of fluid volume data directly into a patient's medical record. This may enhance both individual patient care and support research in postoperative best care practices.

Systems and methods of the subject innovation offer a number of advantages over conventional systems and methods. For example, the subject innovation can decrease nurses' workload, freeing them for other higher-expertise tasks. In contrast to the subject innovation, current practice is for nurses to check patient canisters on average 3 times per hour. Additionally, embodiments of the innovation can decrease time-to-response for emergency conditions; early response to events can lead to better outcomes for the patient and lower costs for the hospital. Further, the subject innovation can convey specific, actionable alert notification information to an individual, rather than a generic tone alarm or broadcast alarm, where no one in particular is directly accountable, which addresses the important clinical problem of alarm fatigue. Additionally, the subject innovation offers the capability to wirelessly stream fluid volume and alert data directly to a patient's electronic medical record, which can enhance individual patient care and support research in postoperative best care practices.

While the innovation has been described above in the general context of computer-executable instructions that may run on one or more computers, those skilled in the art will recognize that the innovation also can be implemented in combination with other program modules and/or as a combination of hardware and software. Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system that facilitates monitoring of a chest tube drainage canister, comprising:
a volume sensor that determines a volume of a fluid in the chest tube drainage canister; and
a control component that calculates a flow rate of the fluid into the chest tube drainage canister, wherein the control component compares the volume and the flow rate with one or more predetermined conditions, and wherein the control component provides at least one notification to a healthcare professional when at least one of the predetermined conditions are met.

2. The system of claim 1, wherein the predetermined conditions comprise at least one of a threshold value associated with the volume, an upper threshold value associated with the flow rate, or a lower threshold value associated with the flow rate.

3. The system of claim 1, further comprising a user interface that displays data associated with at least one of the volume or the flow rate.

4. The system of claim 3, wherein the at least one notification is provided based at least in part on contact information received via the user interface.

5. The system of claim 3, wherein the one or more predetermined conditions are based at least in part on alert information received via the user interface.

6. The system of claim 3, wherein the user interface comprises a touch screen.

7. The system of claim 3, wherein the user interface presents data associated with volume or flow rate in graphical form.

8. The system of claim 1, wherein the at least one notification is provided via one or more of an email or a text message.

9. The system of claim 1, wherein the volume sensor determines the volume by measuring a weight of the fluid.

10. The system of claim 1, wherein the chest tube drainage canister, the volume sensor, and the control component are mounted on an intravenous therapy pole.

11. The system of claim 1, further comprising a battery to power one or more of the chest tube drainage canister, the volume sensor, or the control component.

12. The system of claim 9, wherein a notification is provided to the healthcare professional when a power level of the battery falls below a predetermined threshold.

13. The system of claim 9, further comprising a location component that determines a location of the system, wherein the at least one notification comprises the location when the battery is powering to one or more of the chest tube drainage canister, the volume sensor, or the control component.

14. A method of monitoring a chest tube drainage canister, comprising:
receiving contact information associated with a healthcare professional;
monitoring a volume of a fluid in the chest tube drainage canister;
determining a flow rate of the fluid in the chest tube drainage canister;
comparing the volume and the flow rate to one or more predetermined conditions;
determining whether at least one of the one or more predetermined conditions has been met; and
providing at least one notification to the healthcare professional when the at least one of the one or more predetermined conditions has been met, wherein the notification comprises information indicating which predetermined condition has been met.

15. The method of claim 14, wherein monitoring the volume comprises:
measuring the weight of the fluid; and
estimating the volume of the fluid based at least in part on the weight.

16. The method of claim 14, wherein the one or more predetermined conditions are based at least in part on data received via a user interface.

17. The method of claim 14, wherein the one or more predetermined conditions comprise at least one of a threshold value associated with the volume, an upper threshold value associated with the flow rate, or a lower threshold value associated with the flow rate.

18. The method of claim 14, further comprising determining the location of the chest tube drainage canister, wherein the notification comprises the location of the chest tube drainage canister.

19. The method of claim 14, wherein the notification is provided via at least one of an email or a text message.

20. A system for monitoring a chest tube drainage canister, comprising:
a weight sensor that determines a weight of a fluid in the chest tube drainage canister;
a control component that calculates a volume and flow rate of the fluid based at least in part on the weight of the fluid, wherein the control component compares the volume and the flow rate with conditions associated with one or more alerts, and wherein the control component provides at least one notification to a healthcare professional when at least one of the predetermined conditions are met; and
a user interface that displays information associated with one or more of the volume, the flow rate, or the one or more alerts, wherein the user interface facilitates entry of at least one of contact data for the healthcare professional or data associated with the one or more alerts.
